(19) United States
(12) Patent Application Publication
(10) Pub. No.: US 2023/0330027 A1
(43) Pub. Date: Oct. 19, 2023

(54) PHARMACEUTICAL PREPARATION

(71) Applicant: MERCK PATENT GMBH, Darmstadt (DE)

(72) Inventors: Alessandra AMBRUOSI, Darmstadt (DE); Riccardo MANNINI, Roma (IT); Markus RIEHL, Darmstadt (DE); Axel BECKER, Darmstadt (DE)

(73) Assignee: MERCK PATENT GMBH, Darmstadt (DE)

(21) Appl. No.: 18/026,954

(22) PCT Filed: Sep. 15, 2021
(86) PCT No.: PCT/EP2021/075337

§ 371 Date: Mar. 17, 2023

(30) Foreign Application Priority Data
Sep. 18, 2020 (EP) 20196904.5

(51) Int. Cl. A61K 9/20; A61K 31/4745; A61K 47/26; A61K 47/38; A61K 9/00

(52) U.S. Cl. CPC A61K 9/2095; A61K 31/4745; A61K 47/26; A61K 47/38; A61K 9/0053

(57) ABSTRACT

The present invention relates to a solid pharmaceutical preparation of of 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, as well as a method of making same, as well as medical uses thereof.

The present invention relates to a solid pharmaceutical preparation of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, as well as a method of making same, as well as medical uses thereof.

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is an inhibitor of serine/threonine protein kinase ATM (ataxia telangiectasia mutated kinase.

The serine/threonine protein kinase ATM belongs to the PIKK family of kinases having catalytic domains which are homologous with phospho-inositide-3 kinases (PI3 kinase, PI3K). These kinases are involved in a multiplicity of key cellular functions, such as cell growth, cell proliferation, migration, differentiation, survival and cell adhesion. In particular, these kinases react to DNA damage by activation of the cell cycle arrest and DNA repair programmes (DDR: DNA damage response). ATM is a product of the ATM gene and plays a key role in the repair of damage to the DNA double strand (DSB. double strand breaks). Double-strand damage of this type is particularly cytotoxic. ATM inhibitors are being developed for the treatment of cancer.

8-(1,3-Dimethyl-1H-pyrazol-4-yl)-1-(3-fluoro-5-methoxypyridin-4-yl)-7-methoxy-3-methyl-1,3-dihydroimidazo[4,5-c]quinolin-2-one is a potent ATM inhibitor selected for the clinical development. It is disclosed in WO 2016/155884 (Table 2, Example 4). Surprisingly it has been found that such compound exists in the form of two atropisomers, which can be isolated and are beneficially stable.

The atropisomers of 8-(1,3-Dimethyl-1H-pyrazol-4-yl)-1-(3-fluoro-5-methoxypyridin-4-yl)-7-methoxy-3-methyl-1,3-dihydroimidazo[4,5-c]quinolin-2-one are 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one (formula (1)) and 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one (formula (2)) and are depicted below:

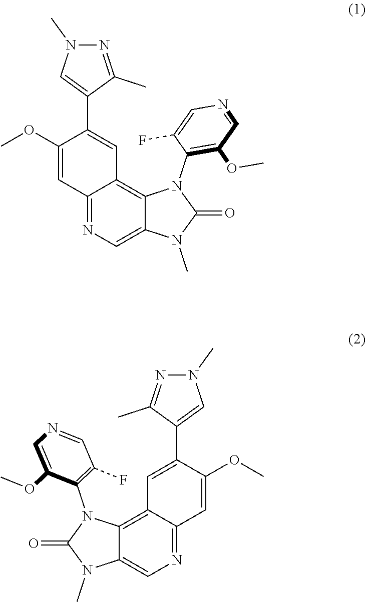

The term “atropisomer” as used herein refers to a stereoisomer which arises due to a restricted rotation around a single bond that creates a chiral axis, whereby the rotation barrier around said single bond has to be sufficiently high to permit the isolation of a single atropisomer. Said rotation barrier can result, for example, from steric interactions with other residues of the same molecule thereby restricting said rotation around said single bond. Both steric and electronic factors come into play and may reinforce or counteract one another.

The utilization of chiral compounds that contain asymmetric carbon atoms is well established in drug discovery, in principle. In particular, it is known in the art that racemic mixtures of two chiral compounds usually consist of one more active and one less active enantiomer as compared to the racemic mixture. Thus, the utilization of only one of the two enantiomers can be advantageous to improve the overall potency of the compound.

However, the utilization of atropisomers, which are stereoisomers that arise only due to a hindered rotation around a single bond, is generally seen as undesirable. In particular, atropisomers are commonly regarded as a liability in drug discovery, since the stability of these isomers depends on energy differences resulting from steric strain or other factors that create a barrier to the rotation around said single bond. In contrast to chiral compounds resulting from asymmetric carbon atoms, atropisomerism cannot be readily predicted. In particular, it is generally not possible to readily predict the stability of an atropisomer. In particular, the height of said energy barrier determines the time of the interconversion of two corresponding atropisomers. The interconversion of a biologically active atropisomer into the corresponding other atropisomer can, thus, reduce its biological activity and introduce off-target or other unwanted effects. Therefore, only stable atropisomers that possess a sufficiently high energy barrier may be suitable in drug discovery. Surprisingly, it has been found that both atropisomers of 8-(1 ,3-Dimethyl-1H-pyrazol-4-yl)-1-(3-fluoro-5-methoxy-pyridin-4-yl)-7-ethoxy-3-methyl-1,3-dihydro-imidazo[4,5-c]quinolin-2-one have sufficient stability and can be used separately for drug development.

Further surprisingly it has further been found that one of the atropisomers, i.e. 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one exhibit especially good properties, which are superior compared to 8-(1,3-Dimethyl-1H-pyrazol-4-yl)-1-(3-fluoro-5-methoxy-pyridin-4-yl)-7-ethoxy-3-methyl-1,3-dihydro-imidazo[4,5-c]quinolin-2-one and 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one, e.g. in terms of its efficacy and selectivity which make it a very suitable candidate for development of a medicament for the treatment of cancer.

The present invention is directed to a solid pharmaceutical preparation of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one.

The invention provides a solid preparation comprising 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or a pharmaceutical acceptable salt thereof and a filler, wherein 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or its pharmaceutical acceptable salt is present from 3 to 90% (w/w) based upon the total weight of the solid preparation. In preferred embodiments 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or its pharmaceutical acceptable salt is present from 5 to 80% (w/w), from 5 to 60% (w/w), 5 to 50% (w/w) 7 to 30% (w/w), 8 to 20% (w/w), exemplary embodiments contain 3, 5, 7, 10, 15, 20, 25, 30, 40, 50, 60, 70, 80 or 90% (w/w).

The solid preparation can comprise 8-(1,3-Dimethyl-1H-pyrazol-4-yl)-1-(Sa)-(3-fluoro-5-methoxy-pyridin-4-yl)-7-methoxy-3-methyl-1,3-dihydro- imidazo[4,5-c]quinolin-2-one in the form of its free base but also in the form of a pharmaceutical acceptable thereof. The term “pharmaceutically acceptable”, as used herein, refers to that which is useful in preparing a pharmaceutical composition that is generally safe, non-toxic, and neither biologically nor otherwise undesirable and includes that which is acceptable for veterinary as well as human pharmaceutical use. The term “pharmaceutically acceptable salt”, as used herein, refers to a salt of a 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one that is pharmaceutically acceptable, as defined herein, and that possess the desired pharmacological activity of the parent 8-(1,3-Dimethyl-1H-pyrazol-4-yl)-1-(Sa)-(3-fluoro-5-methoxy-pyridin-4-yl)-7-methoxy-3-methyl-1,3-dihydro- imidazo[4,5-c]quinolin-2-one. The term “pharmaceutically acceptable salt” includes all hydrates of the respective salt. Appropriate salts may be acid addition salts formed with physiologically acceptable salts, such as, for example, hydrogen halides (for example hydrogen chloride, hydrogen bromide or hydrogen iodide), other mineral acids and corresponding salts thereof (for example sulfate, nitrate or phosphate and the like), alkyl- and monoarylsulfonates (for example ethanedisulfonate (edisylate), toluenesulfonate, napthalene-2-sulfonate (napsylate), benzenesulfonate) and other organic acids and corresponding salts thereof (for example fumarate, oxalate, acetate, trifluoroacetate, tartrate, maleate, succinate, citrate, benzoate, salicylate, ascorbate and the like. Preferred pharmaceutically acceptable salts of 8-(1,3-Dimethyl-1H-pyrazol-4-yl)-1-(Sa)-(3-fluoro-5-methoxy-pyridin-4-yl)-7-methoxy-3-methyl-1,3-dihydro- imidazo[4,5-c]quinolin-2-one that may be present in the solid preparation are its edisylate, fumarate and napsylate salts.

Any reference to amounts or weights or weight percentages of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or pharmaceutically acceptable salts thereof, shall be taken to refer to the anhydrous free form of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, unless specified otherwise herein.

The term “about”, as used herein, refers to a numeric value, including, for example, whole numbers, fractions, and percentages, whether or not explicitly indicated. The term “about” generally refers to a range of numerical values (e.g., +/−1-3% of the recited value) that one of ordinary skill in the art would consider equivalent to the recited value (e.g., having the same function or result). In some instances, the term “about” may include numerical values that are rounded to the nearest significant figure.

As used herein, “a” or “an” shall mean one or more. As used herein when used in conjunction with the word “comprising,” the words “a” or “an” mean one or more than one. As used herein “another” means at least a second or more. Furthermore, unless otherwise required by context, singular terms include pluralities and plural terms include the singular.

As used herein, “%” or “percent” shall mean percent by weight (% (w/w)), unless specified otherwise herein.

The present invention further pertains to a pharmaceutical preparation comprising said solid preparation, methods of preparing the solid preparation and methods of preparing the pharmaceutical preparation, as well as the use of the solid preparation respectively pharmaceutical preparation in the treatment of cancer.

The term “solid preparation”, as used herein, refers to a three-dimensional solid pharmaceutical preparation comprising an active pharmaceutical ingredient (API) and at least one pharmaceutically acceptable excipient. Preferably the solid preparation is a compressed mixture of 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one and one or more pharmaceutically acceptable excipients, for instance selected from a filler and optionally one or more pharmaceutically acceptable excipients. The compressed mixture is obtainable by dry granulation and preferably exists in the form of particles which may have an irregular or regular shape. The solid preparation may be processed to other pharmaceutical preparations such as, for example tablets, but may also be administered to the patient directly without any modification.

The term “filler” as used herein is an agent increasing the bulk of the pharmaceutical preparation by providing the quantity of material which is needed to form a solid preparation. A filler also serves to create desired flow properties and compression characteristics in the preparation of the solid preparation as well as of solid pharmaceutical preparations such as tablets and capsule fillers. Fillers usable in the present invention may be a sugar 25 alcohol such as sorbitol or mannitol, dulcitol, xylitol or ribitol, preferably sorbitol or mannitol, particular preferably mannitol, a sugar such as glucose, fructose, mannose, lactose, saccharose or maltose, preferably lactose, saccharose or maltose, particular preferably lactose, a starch such as potato starch, rice starch, maize starch or pregelatinized starch. Filler can be present in the solid preparation according to the invention in a proportion of 3 to 97% (w/w), preferably 5 to 80% (w/w), particularly preferably to 10 to 50% (w/w), based on the total weight of the solid formulation.

Beside the filler one or more further excipients such as a binder, a glidant, a disintegrant and a lubricant may be present in the solid preparation.

The solid preparation of the present invention comprises 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one in an amount from 3 to 90% by weight based upon the total weight of the solid preparation. According to preferred embodiments 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5- c]quinolin-2-one is present in the solid preparation in an amount from 5 to 50 by weight, more preferred in an amount from 7 to 30% by weight and most preferred in an amount from 8 to 20% by weight based upon the total weight of the solid preparation. Therefore, the invention is also directed to the solid preparation wherein 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5- c]quinolin-2-one is present in an amount from 3 to 90% by weight, preferably from 5 to 50% by weight, more preferably in an amount from 7 to 30% by weight and most preferably in an amount from 8 to 20% by weight based upon the total weight of the solid preparation. In exemplary embodiments of the solid preparation 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5- c]quinolin-2-one is present in an amount of about 3, 5, 7, 10, 15, 20, 25, 30, 40, 50, 60, 70, 80 or 90% (w/w)

According to an preferred embodiment 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is present in the solid preparation as its anhydrous Form A2. Therefore, the invention is also directed to a solid preparation according to claim 1, wherein 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is present as its anhydrous Form A2.

The term “Form A2”, as used herein, refers to a polymorph of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, which has been found to be highly advantageous, being the thermodynamically most stable anhydrous form, and is further exemplified and characterized in the examples, e.g. by way of its powder X-ray diffraction pattern.

Unfortunately in experiments on the development of a solid dosage form Form A2 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one a high stickiness was observed, which lead to problems at its manufacturing and to insufficient content uniformity, thus putting into question its use for drug development. When manufactured by dry granulation using roller compaction, sticking at the rollers questioned the applicability of this manufacturing method. Such tendency could be partially reduced by the addition of a lubricant in the intragranular phase of the granules and by use of a smooth roll instead of a knurled roll but not resolved at all. An alternative approach using fluid bed granulation, which shall reduce the stickiness problems by avoiding sticking at the rollers, could not solve this problem. Further, tablets prepared by using fluid bed granulation lead to higher (thus less desirable) acceptance values for content uniformity. Insufficient content uniformity was obtained although granulates prepared by fluid bed granulation usually have good mixing properties and was obtained even at relatively low drug loadings of 10%. At such a low drug level a person skilled in the art would not expect challenges derived from the drug properties when using fluid bed granulation.

It was surprisingly found, that the solid preparation can be prepared much easier without any sticking problems, if the 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one has a particle size distribution, that is characterized by a d10 value of at least 10 μm, a d50 value of at least 20 μm and a d90 value of not more than 500 μm. Advantageously, the solid preparation prepared with the active pharmaceutical ingredient having such particle size distribution further exhibits improved content uniformity. Further advantageously, the solid preparation prepared with the active pharmaceutical ingredient having such particle size distribution leads to an improved release of the API during in-vitro dissolution testing. Thus, using such solid preparation for a pharmaceutical preparation leads to an improvement of its bioavailability. Therefore, the present invention is also directed to a solid preparation, wherein the particle size distribution of 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is characterized by a d10 value of at least 10 μm, a d50 value of at least 20 μm and a d90 value of not more than 500 μm.

The particle size distribution of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is measured by laser diffraction on a Malvern Mastersizer 2000 (wet method using Hydro 2000S; sample amount of 100 mg dispersed in 5 ml silicone oil; stirrer speed 2000 rpm, no sonication, measuring time of 7.5 s; obscuration of 10-15%). The d values refer to the particle size distribution in micrometers (pm) whereby the dl 0 value refers to the particle diameter in micrometers at which 10 percent of the volume distribution of the particles are smaller than such value, the d50 value refers to the particle diameter in micrometers at which 50 percent of the volume distribution of the particles are smaller than such value and the d90 value refers to the particle diameter in micrometers at which 90 percent of the volume distribution of the particles are smaller than such value.

Advantageously, the ratio between the d90 value and the d10 value is in the range from 7 to 15, preferably from 8 to 14, more preferably from 9 to 13 and is most preferably about 11. Therefore, the present invention is also directed to a solid preparation, whereby the ratio between the d90 value and the d10 value is in the range from 7 to 15, preferably from 8 to 14, more preferably from 9 to 13 and is most preferably about 11.

Further advantageously, the size distribution of the 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one present in the preparation is monomodal. Thus, the present invention is as well directed to a solid preparation according to one or more of claim 1 or 4, wherein 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one present in the preparation has a monomodal particle size distribution.

The term “monomodal” as used herein refers to a particle size distribution having a single relative particle size maximum.

According to a preferred embodiment of the invention the solid preparation comprises as filler a sugar, a sugar alcohol or dicalcium phosphate. According to an especially preferred embodiment the filler is a sugar or a sugar alcohol, whereby the sugar is lactose and the sugar alcohol is sorbitol and/or mannitol, preferably mannitol.

According to a further preferred embodiment of the invention the solid preparation comprises a binder. Thus, the invention is also directed to a solid preparation, wherein the solid preparation further comprises a binder.

The term “binder”, as used herein, refers to an agent that provides cohesion and strength to a solid preparation. Binders which can be employed in the present invention are, for example, polyvinylpyrrolidone, polyvinyl acetate, a vinylpyrrolidone-vinyl acetate copolymer, polyethylene glycol, a starch paste, such as maize starch paste, a cellulose derivative, such as hydroxypropyl methylcellulose, hydroxypropyl cellulose or microcrystalline cellulose, preferably microcrystalline cellulose. Therefore, the present invention is as well directed to a solid pharmaceutical preparation, wherein the binder is polyvinylpyrrolidone, polyvinyl acetate, a vinylpyrrolidone-vinyl acetate copolymer, polyethylene glycol, a starch paste, such as maize starch paste, a cellulose derivative, such as hydroxypropyl methylcellulose, hydroxypropyl cellulose or microcrystalline cellulose, preferably microcrystalline cellulose. Binder can be present in the solid preparation according to the invention in a proportion of 0 to 80% (w/w), preferably 0 to 75% (w/w), particularly preferably to 10 to 60% (w/w), based on the total weight of the solid formulation.

The solid preparation may further comprise a lubricant. Accordingly, one embodiment of the invention is directed to a solid preparation, wherein the solid formulation further comprises a lubricant. The term “lubricant”, as used herein, refers to an inactive ingredient used to prevent sticking of ingredients to one another when dry granulated, filled in capsules or compressed to tablets. A lubricant reduces powder sticking to the roll surface of roller compactors and sliding friction of the tableting material and punches in the die during the tableting operation and prevents sticking to the tablet punches. Suitable lubricants are alkaline-earth metal salts of fatty acids, such as magnesium stearate or calcium stearate, fatty acids, such as stearic acid, higher fatty alcohols such as cetyl alcohol or stearyl alhohol, fats such as glyceryl dipalmitostearate, glyceryl distearate, stearin or glyceryl dibehenate, alkaline-earth metal salts of C16-C18 alkyl substituted dicarbonic acids such as sodium stearyl fumarate, hydrated vegetable oils such as hydrated castor oil or hydrated cotton seed oil, or minerals such as talc. Preferred lubricants are sodium stearyl fumarate, esters of glycerol with fatty acids, stearic acid or pharmaceutically acceptable salts of stearic acid and divalent cations, preferably magnesium stearate. Lubricants can be present in the solid preparation according to the invention in a proportion of 0 to 5% (w/w), preferably 0 to 4% (w/w), particularly preferably 0.25 to 3% (w/w), most preferably about 2% (w/w), based on the total weight of the solid formulation.

The solid preparation may further comprise a disintegrant. Thus, the invention is further directed to a solid preparation, wherein the solid formulation further comprises a disintegrant. The term “disintegrant”, as used herein, refers to a compound that expands and dissolves when wet, to cause disintegration of tablets or granulates to break apart and release the active pharmaceutical agent. The disintegrant also functions to ensure that 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is in contact with the solvent, such as water. Disintegrants serve to disintegrate tablets or granules etc. and thus enhance dissolution of the solid dosage form upon contact with the liquid dissolution medium. Suitable disintegrants include crospovidone (cross linked polyvinyl N-pyrrolidone), cross linked carboxymethylcellulose and salts and derivatives thereof, for instance croscarmellose sodium (cross-linked polymer of carboxymethylcellulose sodium,) sodium carboxymethyl glycolate, sodium starch glycolate, carrageenan, agar, and pectin. Preferred are crospovidone, carboxy starch glycolate, cross linked carboxymethylcellulose or a salt or a derivative thereof, whereby croscarmellose sodium is particularly preferred. Disintegrants are present in the pharmaceutical preparation according to the invention in a proportion of 0 to 20% (w/w), preferably 0.25 to 10% (w/w), particularly preferably 0.5 to 5% (w/w), based on the total weight of the solid formulation.

The solid preparation may further comprise a glidant. Hence, the invention is further directed to a solid preparation, wherein the solid formulation further comprises a glidant. The term “glidant”, as used herein, refers to an inactive ingredient used as a flow aid that improves the flow characteristics of particulates such as powders or granules. In the present invention the glidant improves the flow characteristics of the solid preparation or the mixtures containing the solid preparation during further processing such as encapsulation or tableting. Nonlimiting examples of glidants for use in the present invention include colloidal silicon dioxide (Aerosil 200, Cab-O-Sil), talc, magnesium carbonate, and combinations thereof. Glidants are present in the pharmaceutical preparation according to the invention in a proportion of 0 to 7.5% (w/w), preferably 0 to 5% (w/w), particularly preferably 0 to 3% (w/w), based on the total weight of the solid formulation.

According to an appropriate embodiment of the invention the solid preparation is in the form of particles having a mean particle size that is characterized by a d50 value in the range from 20 μm to 400 μm, preferably from 30 μm to 300 μm and more preferably from 40 to 200 μm. Thus, the invention is also directed to a solid preparation, wherein the solid preparation has a mean particle size that is characterized by a d50 value in the range from 20 μm to 400 μm, preferably from 30 μm to 300 μm and more preferably from 40 to 200 μm.

In order to form a solid preparation dry granulation can be used. The term “dry granulation” or “dry granulating”, as used herein, refers specifically to granulation techniques comprising at least a compaction step. In the pharmaceutical industry two dry granulation methods are primarily used, namely slugging and roller compaction, which both can be used to prepare the solid preparation. Dry granulation by slugging comprises a compaction step using a compression machinery which typically contains two steel punches within a steel die cavity. The granules are formed when pressure is exerted on the material particles by the punches in the cavity and typically have about 25 mm diameter by about 10-15 mm thick, but the particular size of the slug is not a limiting factor for the present invention. Dry granulation by using roller compaction comprises a roller compaction step, wherein material particles are compacted between rotating press rolls, and a subsequent milling step to mill the compacted material into granules. In “dry granulation” processes as usable to prepare the solid preparation, typically, no liquids are employed and/or no drying steps are required. The term “granule” itself does not necessarily imply a specific shape, since the final shape of the granule(s) will be controlled by the specific method of preparation.

The present invention also provides a pharmaceutical preparation comprising the solid preparation according to the invention. Accordingly, the present invention is also directed to a pharmaceutical preparation comprising the solid preparation. The solid preparation may be used as pharmaceutical preparation without any modification but can also be processed to other pharmaceutical preparations such as, for example tablets, or filled into sachets or capsules.

Preferably, the pharmaceutical preparation is for oral administration. Therefore, the present invention is also directed to a pharmaceutical preparation, which is a pharmaceutical preparation for oral administration.

More preferably still, the pharmaceutical preparation is an immediate release preparation. Therefore, the present invention is further directed to pharmaceutical preparation, which is an immediate release preparation.

In exemplary embodiments, the pharmaceutical preparation, preferably a tablet, is characterized by a disintegration time of 30 minutes or less, such as 20 minutes or less, preferably 15 minutes or less, and more preferably 10 minutes or less. The disintegration time referred to above is measured at 37° C. in a disintegration apparatus according to USP-NF <701> (USP39-NF34 Page 537; Pharmacopeial Forum: Volume No. 34(1) Page 155) Disintegration: The apparatus consists of a basket-rack assembly, a 1000-mL, low-form beaker for the immersion fluid, a thermostatic arrangement for heating, and a device for raising and lowering the basket in the immersion fluid. The basket-rack assembly moves vertically along its axis and consists of six open-ended transparent tubes; the tubes are held in a vertical position by two plates. Attached to the under surface of the lower plate is a woven stainless steel wired cloth. If specified in the individual monograph, each tube is provided with a cylindrical disk. The disk is made of a suitable transparent plastic material. One dosage unit is placed in each of the six tubes of the basket and a disk is added. The apparatus is operated and maintained at 37±2° using the specified medium as the immersion fluid. At the end of the time limit or at preset intervals, the basket is lifted from the fluid and observed whether the tablets have disintegrated completely.

In a preferred embodiment, the pharmaceutical preparation according to the present invention is a capsule comprising the solid preparation and optionally one or more pharmaceutically acceptable excipients. The capsule itself may be any pharmaceutically acceptable capsule, such as a hard gelatin capsule, but should preferably be easily dissolvable.

In an exemplary embodiment, the pharmaceutical preparation is a capsule, which contains a mixture consisting of 40 to 100% (w/w), for instance at least 50% (w/w), more preferably at least 70, 80, 90, 95 or 99% (w/w) of the solid preparation according to the present invention; and 0 to 60% (w/w), i.e. the remainder (difference to 100% (w/w)) of the mixture, of at least one pharmaceutically acceptable excipient, preferably selected from a filler, a glidant, a disintegrant and a lubricant, preferably an inorganic alkaline metal salt, more preferably magnesium stearate, based upon the total weight of all material contained in the capsule, i.e. the total weight of the capsule minus the weight of the capsule shell. A preferred embodiment of the invention is directed to pharmaceutical preparation, which is a capsule, which contains 40 to 100% (w/w) of the solid preparation; and 0 to 60% (w/w) of at least one pharmaceutically acceptable excipient, preferably selected from a filler, a glidant, a disintegrant and a lubricant, based upon the total weight of all material contained in the capsule.

In a more preferred embodiment, the pharmaceutical preparation is a tablet, and therefore typically comprises in addition to the pharmaceutically acceptable excipients present in the solid preparation at least one further pharmaceutically acceptable excipient. The at least one additional pharmaceutically acceptable excipient is preferably selected from a filler, a disintegrant, a glidant, a lubricant or a combination thereof. Accordingly, the present invention is also directed to a pharmaceutical preparation, which is a tablet and which in addition to the pharmaceutically acceptable excipients present in the solid preparation optionally comprises one or more pharmaceutically acceptable excipient selected from a filler, a disintegrant, a glidant and a lubricant.

In an exemplary embodiment, the pharmaceutical preparation is a tablet comprising the solid preparation and optionally further excipients, which tablet, based upon its total weight, comprises:
i) 3 to 90% (w/w) of 8-(1,3-Dimethyl-1H-pyrazol-4-yl)-1-(Sa)-(3-fluoro-5-methoxy-pyridin-4-yl)-7-methoxy-3-methyl-1 ,3-dihydro-imidazo[4,5-c]quinolin-2-one or a pharmaceutical acceptable salt thereof;
ii) 3 to 70% (w/w) of a filler;
iii) 0 to 80% (w/w) of a binder;
iv) 0 to 20% (w/w) of disintegrant;
v) 0 to 5% (w/w) of a lubricant;
vi) 0 to 7,5% (w/w) of glidant; and
vii) a total of 0 to 20% (w/w) of one or more additional pharmaceutically acceptable excipients.

The one or more additional pharmaceutically acceptable excipients may include one or more selected from preservatives, antioxidants, sweeteners, flavours, dyes, surfactants, and wicking agents.

Many excipients may exert more than one function, depending on the other components of the pharmaceutical dosage form. For the sake of clarity, in particular in calculating weight percentages, each pharmaceutically acceptable excipient used in a pharmaceutical preparation according to the present invention is preferably associated with one functionality only, i.e. is either regarded as a disintegrant or a lubricant.

In another exemplary embodiment, the pharmaceutical preparation is a tablet comprising the solid preparation and optionally further excipients, which tablet based upon its total weight comprises:
i) 5 to 50% (w/w) of 8-(1,3-Dimethyl-1H-pyrazol-4-yl)-1-(Sa)-(3-fluoro-5-methoxy-pyridin-4-yl)-7-methoxy-3-methyl-1 ,3-dihydro-imidazo[4,5-c]quinolin-2-one or a pharmaceutical acceptable salt thereof;
ii) 5 to 50% (w/w) of a filler;
iii) 0 to 75% (w/w) of a binder;
iv) 0.25 to 10% (w/w) of disintegrant;
v) 0 to 4% (w/w) of a lubricant;
vi) 0 to 5% (w/w) of a glidant; and
vii) a total of 0 to 10% (w/w) of one or more additional pharmaceutically acceptable excipients.

In a further exemplary embodiment, the pharmaceutical preparation is a tablet comprising the solid preparation and optionally further excipients, which tablet based upon its total weight comprises:
i) 7 to 30% (w/w) of 8-(1,3-Dimethyl-1H-pyrazol-4-yl)-1-(Sa)-(3-fluoro-5-methoxy-pyridin-4-yl)-7-methoxy-3-methyl-1 ,3-dihydro-imidazo[4,5-c]quinolin-2-one or a pharmaceutical acceptable salt thereof;
ii) 10 to 30% (w/w) of a filler;
iii) 10 to 60% (w/w) of a binder;
iv) 0.5 to 5% (w/w) of disintegrant;
v) 0.25 to 3% (w/w) of a lubricant;
vi) 0 to 3% (w/w) of a glidant; and
vii) a total of 0 to 10% (w/w) of one or more additional pharmaceutically acceptable excipients.

Preferably, in those embodiments, the filler is mannitol, the binder is microcrystalline cellulose, the disintegrant is selected from crospovidone, carboxy starch glycolate, cross linked carboxymethylcellulose and salts and derivatives thereof, especially croscarmellose sodium, the lubricant is selected from magnesium stearate, calcium stearate and sodium stearyl fumarate, preferably magnesium stearate, and/or the glidant is selected from colloidal silicon dioxide and derivatives thereof. In an especially preferred embodiment the filler is mannitol, the binder microcrystalline cellulose, the disintegrant is croscarmellose sodium, the lubricant is magnesium stearate and the glidant is colloidal silicon dioxide.

Preferably, the total of one or more additional pharmaceutically acceptable excipients is 0 to 10% (w/w), 0 to 7.5% (w/w), 0 to 5% (w/w), 0 to 2.5% (w/w) or 0 to 1% (w/w), for instance 0% (w/w).

Of course, the tablet may be coated, to improve taste and/or appearance and/or to protect the tablet from external influences such as moisture. Any coating shall not count towards the total of 100% (w/w) of pharmaceutically active ingredients and drug substance making up the tablets, as listed above. For film-coating, macromolecular substances, such as modified celluloses, including hydroxypropyl methylcellulose (HPMC), polyvinyl alcohol (PVA) such as, for example, with polyethylene glycol (PVA-PEG copolymer), polymethacrylates, polyethylene glycols, and zein may be used, for example. The thickness of the coating is preferably less than 200 μm.

The present invention also provides a method for preparing the solid preparation, which comprises dry granulating, such as slugging and roller compaction, preferably roller compaction. Accordingly, the present invention is also directed to a method for preparing the solid preparation, the method dry granulating, preferably roller compacting.

The term “roller compaction” or “roller compacting” refers to a process in which powders or particles are forced between two counter rotating rolls and pressed into a solid compact or ribbon. Roller compacting can be carried out with any suitable roller compactor known to the skilled person. Suitable roller compactors include, for example, a Fitzpatrick® Chilsonator IR220 roller compactor of the Fitzpatrick Company, USA. The process parameters, especially the roll force, can be readily accomplished by routine experimentation based upon the common general knowledge of the person skilled in the art. Suitable roll force may be, for example, in the range from 2 to 16 kN/cm, more preferably in the range from 4 to 12 kN/cm and most preferably in the range from 4 to 8 kN/cm.

In an exemplary embodiment, the method comprises:
(a) mixing 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or a pharmaceutical acceptable salt thereof, and a filler and optionally one or more further pharmaceutically acceptable excipient;
(b) dry granulating the mixture prepared by step (a) to form the solid preparation; and
(c) optionally milling.

Preferred pharmaceutical acceptable excipients used in step (a) are selected from a binder, a disintegrant, a lubricant and a glidant. According to a preferred embodiment, dry granulating used in the method is roller compacting.

The solid preparation prepared can be used for the preparation of pharmaceutical preparations such as tablets or capsules. An exemplary method for preparing a pharmaceutical preparation, which is a tablet, comprising the solid preparation, comprises
(a) conducting the method described above to form the solid preparation;
(b) mixing the solid preparation and one or more pharmaceutically acceptable excipients;
(c) tableting the mixture prepared by step (b); and
(d) optionally film coating of the tablets prepared by step (c).

Tableting respectively compressing into tablets can be performed with commonly used eccentric presses or rotary presses.

An exemplary method for preparing a pharmaceutical preparation, which is a capsule, comprising a solid preparation, comprises
(a) conducting the method to form the solid preparation;
(b) optionally mixing the solid preparation and one or more pharmaceutically acceptable excipient and optionally granulating the mixture obtained, preferably by roller compaction;
(c) filling the mixture or granulate prepared by step (b) or the solid preparation prepared by step (a) into capsules.

As set out above in the introductory section, 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one has been found to exhibit valuable properties as an ATM inhibitor that finds application in the treatment of cancer. It is intended to be investigated in clinical trials.

Accordingly, the present invention provides the solid preparation respectively pharmaceutical preparation as described above, for use in the treatment of cancer.

Optionally the treatment of cancer further comprises radiotherapy. Accordingly, the present invention is also directed to the pharmaceutical preparation of the present invention for use in the treatment of cancer optionally together with radiotherapy.

Optionally, in the alternative or in addition to radiotherapy, the treatment of cancer may comprise chemotherapy. Accordingly, the present invention is also directed to the pharmaceutical preparation for use in the treatment of cancer.

In an exemplary embodiment, the present invention provides a method of treating solid cancers, in a patient in need thereof, comprising administering to said patient 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one.

In the following, the present invention will be described by reference to exemplary embodiments thereof, which shall not be regarded as limiting the invention.

### BRIEF DESCRIPTION OF THE FIGURES

FIG. 1 Shows typical Particles Size Distribution profiles obtained on Compound 1 Anhydrous Form A2 (upper panel) and Compound 1 Anhydrous Form A2 OPT (lower panel).

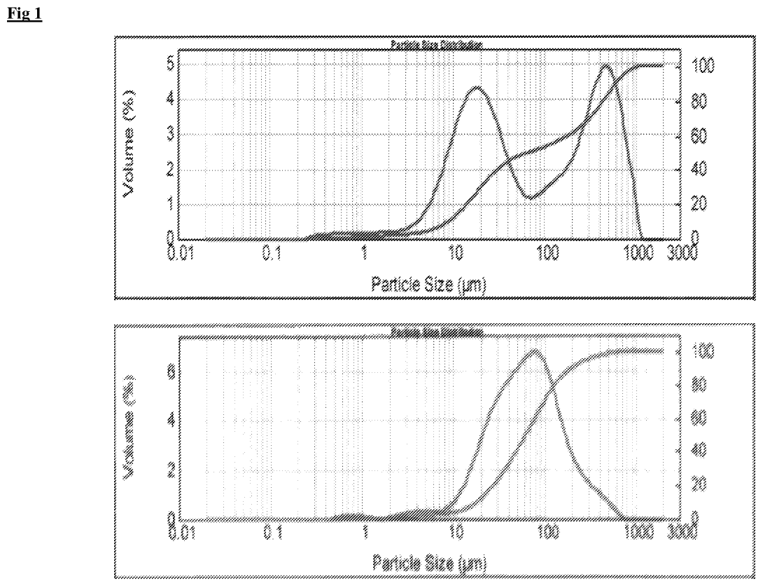

FIG. 2 shows dissolution curves obtained for roller compaction prototypes (black curve, black circles: Preparation A, as per Example 15; grey curve, grey circles: Preparation B, as per Example 14; black curve, white circles: Final Preparation, still referring to Example 14), showing the consistent improvement in dissolution levels obtained by replacing Compound 1 Anhydrous Form A2 with Compound 1 Anhydrous Form A2 OPT). Dissolution conditions are the following: 900 mL phosphate buffer, pH=6.8 using a paddle apparatus with 75 rpm, 37° C.

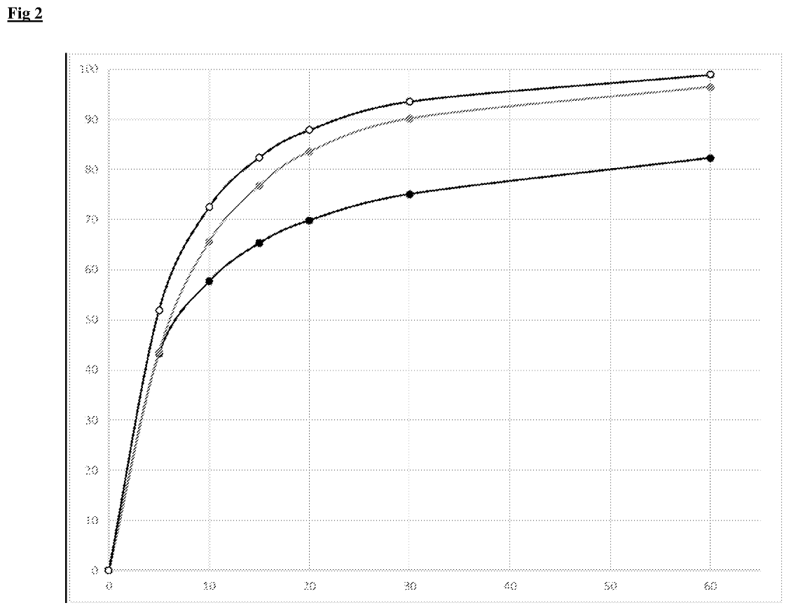

FIG. 3 shows the X-Ray diffractogram of solid Compound 1 Anhydrous Form A2 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one material.

### ACTIVE PHARMACEUTICAL INGREDIENT

### Example 1

8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one is prepared in accordance with the procedure disclosed in WO 2016/155844, followed by separation of the atropisomers, as illustrated by the following reaction scheme:

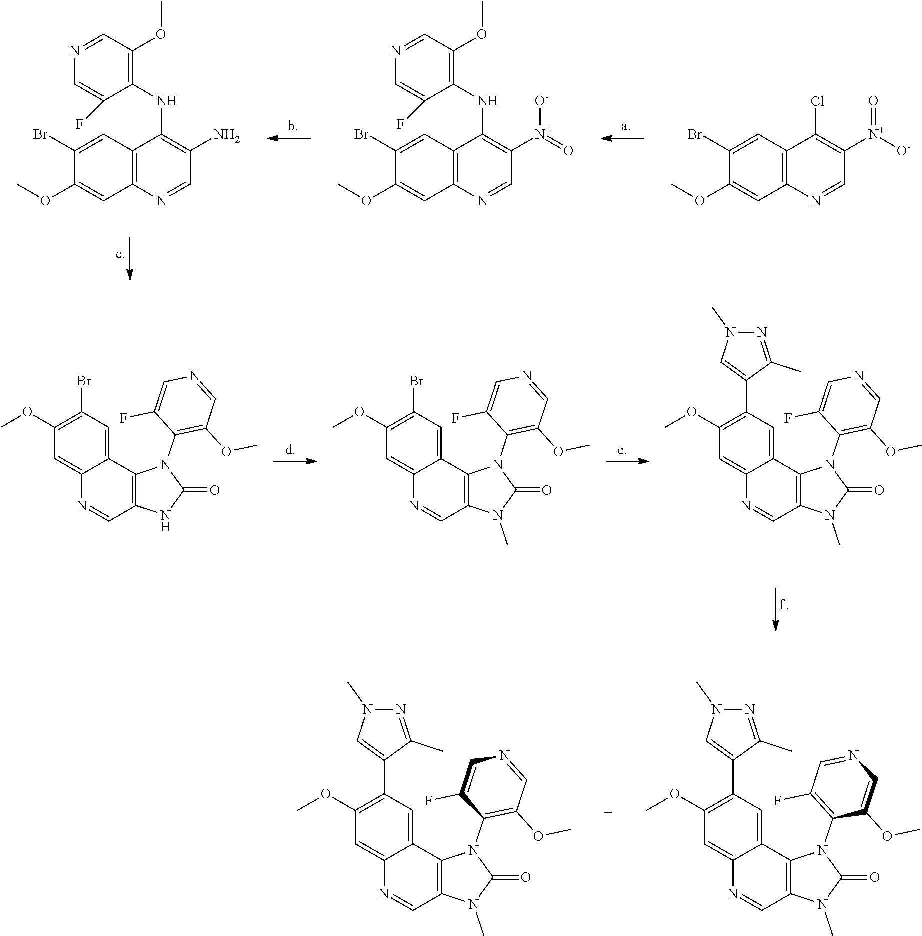

a. Synthesis of 6-bromo-N-(3-fluoro-5-methoxy-4-pyridyI)-7-methoxy-3-nitro-quinolin-4-amine
   Under a dry nitrogen atmosphere, a solution of 3-fluoro-5-methoxypyridin-4-amine (447 mg, 3.02 mmol) dissolved in N,N-dimethylformamide (5 mL) is provided. Then, sodium hydride (504 mg, 12.6 mmol, 60%) is added to the solution and stirring continued for 5 minutes at room temperature. 6-Bromo-4-chloro-7-methoxy-3-nitro-quinoline (800 mg, 2.52 mmol) is then added to the reaction mixture, followed by 15 minutes of stirring at room temperature, then by quenching of the reaction through addition of ice water (100 mL). The precipitate is filtered off, washed with ice water and dried to give 1.00 g (94%) 6-bromo-N-(3-fluoro-5-methoxy-4-pyridyI)-7-methoxy-3-nitro-quinolin-4-amine as a yellow solid.
b. Synthesis of 6-bromo-N4-(3-fluoro-5-methoxy-4-pyridyl)-7-methoxy-quinoline-3,4-diamine:
   6-Bromo-N-(3-fluoro-5-methoxy-4-pyridyI)-7-methoxy-3-nitro-quinolin-4- amine (990 mg, 2.20 mmol) dissolved in methanol (100 mL) is provided under a protective nitrogen atmosphere. Then, Raney-Ni (100 mg, 1.17 mmol) is added to the solution, and the reaction mixture is stirred for 30 minutes under a hydrogen atmosphere at normal pressure. After introducing nitrogen, the suspension is filtered and the filtrate dried under vacuum. The filtrate is evaporated to dryness under vacuum. The residue is crystallized from a mixture of ethyl acetate/petroleum ether, yielding 0.86 g (99%) 6-bromo-N⁴-(3-fluoro-5-methoxy-4-pyridyI)-7-methoxy-quinoline-3,4-diamine as a yellow solid.
c. Synthesis of 8-bromo-1-(3-fluoro-5-methoxy-4-pyridyI)-7-methoxy-3H-imidazo[4,5-c]quinolin-2-one
   A solution of 6-bromo-N⁴-(3-fluoro-5-methoxy-4-pyridyI)-7-methoxy-quinoline-3,4-diamine (0.85 g, 2.20 mmol) dissolved in tetrahydrofuran (20 mL) is provided. Then, 1,1′-carbonyldiimidazole (1.84 g, 11.3 mmol) and Hünig's-base (1.46 g, 11.3 mmol) were added. The reaction mixture is heated to 40° C. and stirred for 16 hours. The reaction is then quenched by the addition of ice water (200 mL). The precipitate is filtered off, washed with ice water and dried to give 0.87 g (94%) 8-bromo-1-(3-fluoro-5-methoxy-4-pyridyl)-7-methoxy-3H-imidazo[4,5-c]quinolin-2-one as a light yellow solid.
d. Synthesis of 8-bromo-1-(3-fluoro-5-methoxy-4-pyridyI)-7-methoxy-3-methyl-imidazo[4,5-c]quinolin-2-one:
   In a dry protective nitrogen gas atmosphere, 8-bromo-1-(3-fluoro-5-methoxy-4-pyridyl)-7-methoxy-3H-imidazo[4,5-c]quinolin-2-one (0.86 g, 1.94 mmol) dissolved in N,N-dimethylformamide (5 mL) is provided. Then, sodium hydride (388 mg, 9.71 mmol, 60%) and methyl iodide (2.76 g, 19.4 mmol) were added. The reaction mixture is stirred for 10 minutes at room temperature. Then the reaction is quenched by the addition of ice water (100 mL). The resulting precipitate is filtrated and dried under vacuum to give 0.70 g (80%) 8-bromo-1-(3-fluoro-5-methoxy-4-pyridyI)-7-methoxy-3-methyl-imidazo[4,5-c]quinolin-2-one as a light yellow solid.

e. Synthesis of 1-(3-fluoro-5-methoxy-4-pyridyl)-7-methoxy-3-methyl-8-(1,3-dimethylpyrazol-4-yl)imidazo[4,5-c]quinolin-2-one:

Under an argon inert gas atmosphere in closed equipment 8-bromo-1-(3-fluoro-5-methoxy-4-pyridyl)-7-methoxy-3-methyl-imidazo[4,5-c]quinolin-2-one (150 mg, 0.33 mmol), 1-3-dimethyl-4-(tetramethyl-1,3,2-dioxaborolan-2-yl)-1H-pyrazole (88.4 mg, 0.40 mmol), Pd(PPh₃)₄ (76.6 mg, 0.07 mmol) and potassium carbonate (91.6 mg, 0.66 mmol) in 1,4-dioxane (15 mL) and water (5 mL) are provided. The reaction mixture is heated to 80° C. with stirring for 2 hours. This is followed by cooling to room temperature and reducing the reaction mixture to dryness under vacuum. The residue is chromatographically purified using silica (ethyl acetate/methanol =97:3, parts by volume). The eluate is reduced to dryness and the resulting raw product purified by means or preparative RP-HPLC (water/acetonitrile). After reducing the product fractions, 1-(3-fluoro-5-methoxy-4-pyridyI)-7-methoxy-3-methyl-8-(1,3-dimethylpyrazol-4-yl)imidazo[4,5-c]quinolin-2-one (70 mg, 47%) is obtained as a colourless solid.

f. Separation of 8-(1,3-dimethyl-1H-pyrazol-4-yl)-1-(Ra)-(3-fluoro-5-methoxy-pyridin-4-yl)-7-methoxy-3-methyl-1,3-dihydro-imidazo[4,5- c]quinolin-2-one and 8-(1,3-dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one:

1-(3-fluoro-5-methoxy-4-pyridyI)-7-methoxy-3-methyl-8-(1,3-methylpyrazol-4-yl)imidazo[4,5-c]quinolin-2-one (50.0 mg, 0.11 mmol) as obtained above is separated via chiral HPLC using SFC to give Compounds 1 and 2. The substance is applied to chiral column Lux Cellulose-2 and separated at a flow of 5 mL/min with CO₂/2-propanol+0.5% diethylamine (75:25) as the solvent and using detection at a wavelength of 240 nm. Reducing the product fractions at reduced pressure yielded 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one (25.0 mg, 50%) and 8-(1,3- Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one (22.1 mg, 44%), both as colorless solids.

The starting compounds for the above reactions are readily obtainable, for instance as shown below:

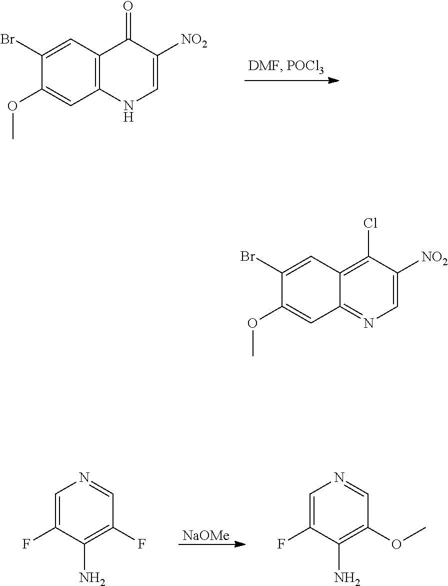

The atropisomers can be isolated from the first compound using chromatography on a chiral stationary phase (see, e.g., Chiral Liquid Chromatography; W. J. Lough, Ed. Chapman and Hall, New York, (1989); Okamoto, “Optical resolution of dihydropyridine enantiomers by high-performance liquid chromatography using phenylcarbamates of polysaccharides as a chiral stationary phase”, J. of Chromatogr. 513:375-378, (1990)). 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one and 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one can be isolated by chromatography on chiral stationary phase, for example, a Chiralpak IC column (5 mm, 150×4.6 mm I.D.) e.g., using isocratic elution with a mobile phase containing: H₂O/ACN 50/50 v/v (ACN: acetonitrile; v: volume). A suitable chromatogram may be obtained using the following conditions: Column and elution as mentioned above, flow 1.00 ml/min; UV @ 260 nm; T_{c} and T_{S}: 25±5° C., S_{conc} 0.20 mg/ml; injected volume 10 ml.

As an alternative to the SFC conditions mentioned above, preparative supercritical fluid chromatography may be used, involving for instance: Chiralpak AS-H (20 mm×250 mm, 5 μm) column; isocratic elution (20:80 ethanol:CO₂ with 0.1% v/v NH₃), BPR (back-pressure reg.): about 100 bar above atmospheric pressure; a column temperature of 40° C., a flow rate of 50 ml/min, an injection volume of 2500 μl (125 mg) and a detector wavelength of 265 nm, with the (Sa)-atropisomer eluting second (after the (Ra)atropisomer)).

For the analysis of the purity of the respective atropisomers, again, SFC may be applied, for instance using the following set-up: Chiralpak AS-H (4.6 mm×250 mm, 5 μm) column; isocratic elution (20:80 ethanol:CO₂ with 0.1% v/v NH₃), BPR (back-pressure reg.): about 125 bar above atmospheric pressure; a column temperature of 40° C., a flow rate of 4 ml/min, an injection volume of 1 μl and a detector wavelength of 260 nm.

The atropisomers may also be isolated from the first compound through preparation of chiral salts, for instance using dibenzoyl-L-tartaric acid, as illustrated in the scheme below:

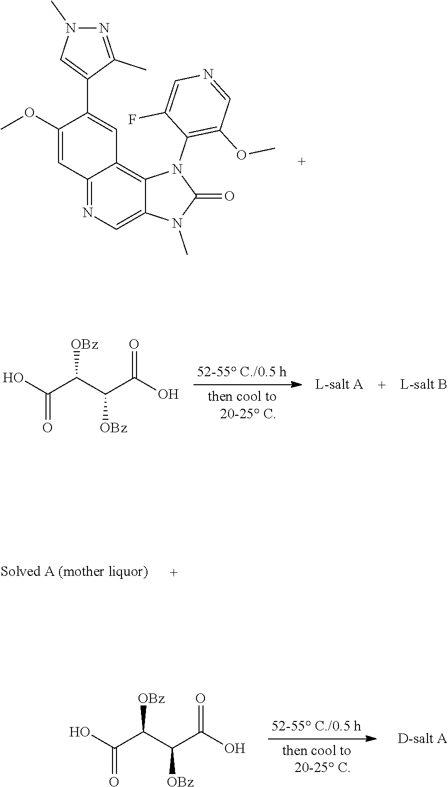

It has been found that 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one inhibits the ATM pCHK2 pathway at lower concentrations and has a better selectivity compared to 8-(1,3-Dimethyl-1H-pyrazol-4-yl)-1-(Ra)-(3-fluoro-5-methoxy-pyridin-4-yl)-7-methoxy-3-methyl-1,3-dihydro- imidazo[4,5-c]quinolin-2-one and 8-(1,3-Dimethyl-1H-pyrazol-4-yl)-1-(3-fluoro-5-methoxy-pyridin-4-yl)-7-ethoxy-3-methyl-1,3-dihydro-imidazo[4,5-c]quinolin-2-one. Surprisingly, it does not only have the best ATM inhibiting properties, but also best microsomal clearance values and lowest inhibition of phosphodiesterase (PDE) 2A1 as well as PDE4A1A and PDE4D2. Overall, 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one has been found to have the most beneficial overall combination of properties and is especially suitable for the drug development.

### Example 2

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one can be obtained and isolated as solid as hydrate or in its anhydrous form.

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one hydrate can exist in two polymorphic forms, Form H1 and Form H2, which can be obtained individually by recrystallization in different solvents. For example,

Form H1 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one hydrate can be obtained, if recrystallized in methanol, and Form H2 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one hydrate can be obtained, if recrystallized in water.

Anhydrous 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one can exist in three polymorphic forms, Form A1, Form A2 and Form A3, which can be obtained individually by recrystallization in different solvents.

It has been found that Anhydrous Form A2 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is highly advantageous, being the thermodynamically most stable anhydrous form, and therefore, the preferred form for development. Accordingly, the solid preparation of the present invention comprises 8-(1,3-Dimethyl-1H-pyrazol -4-yl)-1-(Sa)-(3-fluoro-5-methoxy-pyridin-4-yl)-7-methoxy-3-methyl-1,3-dihydro-imidazo[4,5-c]quinolin-2-one preferably in its Anhydrous Form A2.

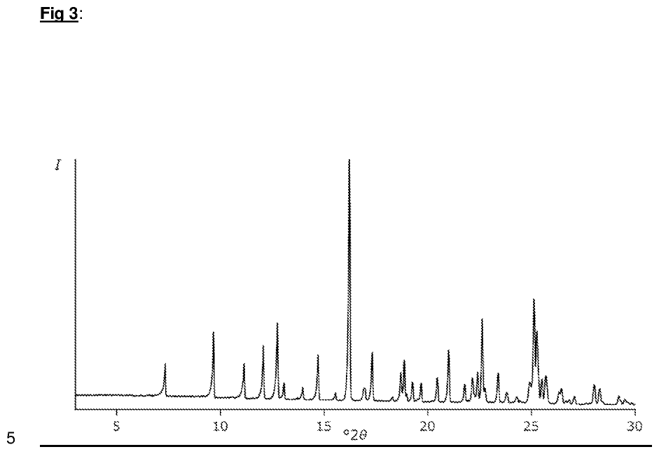

X-ray powder diffraction (XRPD) pattern of solid Anhydrous Form A2 of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one present is shown in FIG. 3.

Accordingly, Anhydrous Form A2 is characterized by one or more peaks in its powder X-ray diffraction pattern selected from those at about 7.3, about 9.6, about 11.1, about 12.0, about 12.7, and about 16.2 degrees 2-theta. In some embodiments, Anhydrous Form A2 is characterized by two or more peaks in its powder X-ray diffraction pattern selected from those at about 7.3, about 9.6, about 12.7, about 16.2, about 22.6 and about 25.1 degrees 2-theta. In certain embodiments, Anhydrous Form A2 is characterized by three or more peaks in its powder X-ray diffraction pattern selected from those at about 7.3, about 9.6, about 12.7, about 16.2, about 22.6 and about 25.1 degrees 2-theta. In certain embodiments, Anhydrous Form A2 is characterized by substantially all of the peaks in its powder X-ray diffraction pattern selected from those at about 7.3, about 9.6, about 12.7, about 16.2, about 22.6 and about 25.1 degrees 2-theta. In particular embodiments, Anhydrous Form A2 is characterized by substantially all of the peaks in its X-ray powder diffraction pattern selected from those at about 7.3, 9.6, 11.1, 12.0, 12.7, 14.7, 16.2, 17.3, 18.9, 21.0, 22.6 and 25.1 degrees 2-theta.

Anhydrous Form A2 can be obtained from 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one by cooling crystallisation from e.g. ethyl acetate or alcohols. For example, Anhydrous Form A2 can be obtained following the method and reaction conditions as described in the following in Examples A, B and C.

### Example A

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is dissolved in 1-propanol at a concentration of approx. 50 mg/mL at 50° C. under stirring. The resulting clear solution is cooled to −20° C. at a cooling rate of 0.1 ° C/min., with a final hold period of at least 1 h at −20° C. Solid-liquid separation is performed by filtration over vacuum suction, and the filtrated l solid sample is dried under dynamic nitrogen purge overnight.

### Example B

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one is dissolved in iso-butylalcohol at a concentration of approx. 40 mg/mL at 50° C. under stirring. The resulting clear solution is cooled to −20° C. at a cooling rate of 0.1° C/min., with a final hold period of at least 1 h at −20° C. Solid-liquid separation is performed by filtration over vacuum suction, and filtrated solid sample is dried under dynamic nitrogen purge overnight.

### Example C

8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, hydrate form H2 [as obtained from racemic resolution step of Sₐ and Rₐ atropisomers] is dispersed in ethyl acetate at a concentration of approx. 190 mg/mL. The dispersion is agitated at RT (20-25° C.) for 21 hours, filtrated via vacuum-suction, and dried in a vacuum drying cabinet at 60° C. for 72 hours.

In the following different forms of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one are used. If 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one prepared in accordance to Example 1 is used, it is referred to “Compound 1”. If 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one anhydrous Form A2 as it is obtainable using the proceeding described in Example A, Example B or Example C used, it is also referred to as “Compound 1 Anhydrous Form A2”.

### Preformulation Examples

In initial preformulation studies with Compound 1 Anhydrous Form A2 manufacturing of tablet prototypes was afflicted with undesired sticking phenomena and led somewhere to unacceptable low content uniformity of tablets after coating and, when tested, an unsatisfactory level of dissolution.

Table 1 shows an example of the main results obtained at that early stage of development (when dissolution was not yet present in the testing panel), together with the formulation composition of the relevant prototypes.

Prototype I was prepared as described in EXAMPLE 1, Prototypes II and III as described in EXAMPLE 2-3 of the section Formulation Examples hereinafter.

TABLE 1

| Dosage Strengths | | | |
|---|---|---|---|
| vs. Substances | | | |
| [%/tablet] | Prototype I | Prototype II | Prototype III |
| Compound 1 | 10% | 10% | 10% |
| Lactose | 43.5% | 43.5% | — |
| Microcrystalline | 43.5% | 43.5% | 43.5% |
| Cellulose (MCC) | | | |
| Mannitol | — | — | 43.5% |
| Croscarmellose | 1.0% | | 1.0% |
| Crospovidon | — | 1.0% | — |
| Magnesium | 1.0% | 1.0% | 1.0% |
| stearate | | | |
| Silicon dioxide | 1.0% | 1.0% | 1.0% |

Analytical Results

| | | | |
|---|---|---|---|
| Disintegration time, | 01:06 | 00:56 | 01:43 |
| max. [mins] | | | |
| Assay of tablets | 10.2 | 10.7 | 10.2 |
| cores [mg/tablet; | | | |
| average] | | | |
| Content Uniformity | 5.3 | 8.7 | 4.5 |
| of tablets cores | | | |
| [acceptance value] | | | |

Content Uniformity test and calculation of acceptance values were performed in accordance with the indications of Section 2.9.40 of European Pharmacopeia. The lower the acceptance value the better is the content uniformity. Disintegration time was measured in accordance to Section 2.9.1 of European Pharmacopeia.

Assay and identity of the mentioned solid pharmaceutical preparations are tested by high-performance liquid chromatography with UV detection using a reversed phase column and an isocratic system, after preparation and during the stability studies. The extraction medium and mobile phase used are mixtures of acetonitrile, water and trifluoroacetic acid.

Actually the preliminary results, obtained on tablets cores (no coating applied yet), looked as promising; on the other hand, sticking phenomena observed during manufacturing required some improvement of the process.

Having the tendency to agglomeration, observed when preparing Compound 1 Anhydrous Form A2 with final slurry in ethyl acetate, been identified as potential cause for poor performance during tablets prototypes manufacturing, the following optimized preparation process has been then put in place for Compound 1:
Hot solution in 2-Propanol is prepared for Compound 1 hydrate form
H2 at 80° C. at a concentration level of 12% w/w (based on dry mass of compound 1).
The obtained hot solution is cooled down to 70° C.
The obtained hot solution is held at 70° C., to implement a seeding step (seeding with target morphic form A2), and thus control particle properties.
The seeded supersaturated solution is cooled down to 5° C. with a linear cooling rate of 0.1 K/min.
Finally, the obtained suspension is slurry-aged at 5° C. for at least 1 h, followed by solid/liquid separation (vacuum filtration), washing, and drying of isolated solid material at 70° C. for at least 10 h.

It has to be noted that, for seeding step, medium-sized seed crystals 20-40 μm are used, obtained from preparative sieving step of Compound 1 Anhydrous Form A2 from initial 2-Propanol-based crystallization trials using 20 μm and 40 μm mesh sieves, using manual compaction force to press larger particles through upper 40 μm mesh and eliminating small particle fraction <20 μm by subsequent high-frequency shaking of lower 20 μm sieve; the seed crystal quantity is 4.5% w/w (related to the target amount of compound 1 in solution).

Compound 1 Anhydrous Form A2 as it is obtainable from such preparation process is hereinafter referred as “Compound 1 Anhydrous Form A2 OPT”

Unexpectedly it has been found that the sticking phenomena can be avoided and tablets having an improved content uniformity and dissolution properties can be prepared, while exhibiting comparable behavior for other parameters, if the tablet is manufactured with Compound 1 Anhydrous Form A2 OPT instead of Compound 1 Anhydrous Form A2 (see Table 2 showing the data of two preparations, which have been prepared by using the same process and the same auxiliaries but which differ from each other in that that one of these preparations contains as API Compound 1 Anhydrous Form A2 and the other Compound 1 Anhydrous Form A2 OPT).

TABLE 2

| | Preparation A | Preparation B |
|---|---|---|
| | (ref. Example | (ref. Example |
| | 15) | 14) |
| Compound 1 Anhydrous Form A2 | 10.00% | — |
| Compound 1 Anhydrous Form A2 OPT | — | 10.00% |
| Microcrystalline Cellulose (MCC 101) | 51.70% | 51.70% |
| Mannitol M100 | 23.30% | 23.30% |
| Croscarmellose sodium | 1.00% | 1.00% |
| Magnesium stearate | 0.50% | 0.50% |
| Silicon dioxide | 1.00% | 1.00% |
| Silicon dioxide | 1.00% | 1.00% |
| Magnesium stearate | 1.50% | 1.50% |
| Microcrystalline Cellulose (MCC 101) | 10.00% | — |
| Microcrystalline Cellulose (MCC 102) | — | 10.00% |

Analytical results

| | | |
|---|---|---|
| Content Uniformity [Acceptance Value | 8.2 | 3.2 |
| correlated to standard deviation] | | |
| Dissolution level after 30 minutes [%] | 76.5 | 90.1 |

Table 3 shown hereinafter provides further evidence of the reached improvement, details on the final composition reached at the end of formulation development, together with the main analytical results obtained on the corresponding tablets batch, are summarized in the table hereafter.

TABLE 3

| Dosage Strengths vs. | Final Preparation |
|---|---|
| Substances [mg/tablet] | (as per Example 14) |
| Compound 1 Anhydrous Form A2 OPT | 50.00 |
| MCC 101 | 258.50 |
| Mannitol M100 | 116.50 |
| Croscarmellose sodium | 5.00 |
| Magnesium stearate | 2.50 |
| Silicon dioxide | 5.00 |
| Silicon dioxide | 5.00 |
| Magnesium stearate | 7.50 |
| MCC 102 | 50.00 |
| Coating; | 15.00 |
| Polyvinyl alcohol polyethylene glycol | |
| (PVA-PEG) copolymer (Opadry QX) | |

Analytical Results

| | |
|---|---|
| Assay [mg/tablet; average] | 49.73 |
| Content Uniformity [acceptance value] | 2.8 |
| Dissolution level after 30 minutes [%] | 93.6 |

As apparent from table 3 the preparation has a very good Content Uniformity and provides an excellent dissolution level after 30 minutes.

Table 4 shows the representative particle size distribution (in terms of key values and obtained profile) of Compound 1 Anhydrous Form A2 versus Compound 1 Anhydrous Form A2 OPT. As apparent therefrom the improved manufacturing properties as well as the improved properties of the preparation containing of Compound 1 Anhydrous Form A2 OPT versus Compound 1 Anhydrous Form A2 can be attributed to their different particle size distributions.

TABLE 4

| | Compound 1 Anhydrous | Compound 1 Anhydrous |
|---|---|---|
| | Form A2 | Form A2 OPT |
| Dv10 | 9 | 19 |
| Dv50 | 63 | 64 |
| Dv90 | 599 | 202 |

### FORMULATION EXAMPLES

### Exemplary Solid Preparation Formulations for dry granulation

### Example 1

The ingredients are weighed (batch size of 500 g) and sieved through a 1.0 mm sieve. The blend is produced by mixing all ingredients in a commercially available bin blender (e.g. Limitec) for 10 min with 10 rpm. The mixture is transferred afterwards to a roller compactor for manufacturing of the solid preparation. The roller compactor (Hosokawa Alpine) is run with the following settings: Compaction force 5 kN/cm, gap width 1.5 mm, roll speed 3.0 rpm. The resulting granules are sieved through a 0.8 mm sieve.

### Example 2-3

The ingredients are weighed (batch size of 500 g) and sieved through a 1.0 mm sieve. The blend is produced by mixing all ingredients except magnesium stearate in a commercially available bin blender (e.g. Limitec) for 10 min with 10 rpm. The magnesium stearate is added afterwards and the whole mixture is blended again for 3 min with 10 rpm. The mixture is transferred afterwards to a roller compactor for manufacturing of the solid preparation. The roller compactor (Hosokawa Alpine) is run with the following settings: Compaction force 5 kN/cm, gap width 1.5 mm, roll speed 3.0 rpm. The resulting granules are sieved through a 0.8 mm sieve.

### Examples 4

The ingredients are weighed (batch size of 500 g) and sieved through a 1.0 mm sieve. The blend is produced by mixing all ingredients except magnesium stearate in a commercially available bin blender (e.g. Limitec) for 10 min with 10 rpm. The magnesium stearate is added afterwards and the whole mixture is blended again for 3 min with 10 rpm. The mixture is transferred afterwards to a roller compactor for manufacturing of the solid preparation. The roller compactor (Hosokawa Alpine) is run with the following settings: Compaction force 3 kN/cm, gap width 1.5 mm, roll speed 3.0 rpm. The resulting granules are sieved through a 0.8 mm sieve.

### Example 5

The ingredients are weighed (batch size of 500 g) and sieved through a 1.0 mm sieve. The blend is produced by mixing all ingredients except magnesium stearate in a commercially available bin blender (e.g. Limitec) for 10 min with 10 rpm. The magnesium stearate is added afterwards and the whole mixture is blended again for 3 min with 10 rpm. The mixture is transferred afterwards to a roller compactor for manufacturing of the solid preparation. The roller compactor (Hosokawa Alpine) is run with the following settings: Compaction force 3 kN/cm, gap width 2.0 mm, roll speed 3.0 rpm. The resulting granules are sieved through a 0.8 mm sieve.

### Example 6-7

The ingredients are weighed (batch size of 15 Kg) and sieved through a 1.0 mm sieve. The blend is produced by mixing all ingredients except magnesium stearate in a commercially available bin blender (e.g. Limitec) for 10 min with 10 rpm. The magnesium stearate is added afterwards and the whole mixture is blended again for 3 min with 10 rpm. The mixture is transferred afterwards to a roller compactor for manufacturing of the solid preparation. The roller compactor (Hosokawa Alpine) is run with the following settings: Compaction force 7 kN/cm, gap width 2.0 mm, roll speed 3.0 rpm. The resulting granules are sieved through a 0.8 mm sieve.

| Solid | | |
|---|---|---|
| prepa- | | |
| ration # | Composition | % (w/w) |
| 1 | Solid preparation consisting of | |
| | 8-(1,3-Dimethyl-1H-pyrazol-4-yl)-1-(3-fluoro-5- | 10.00 |
| | methoxy-pyridin-4-yl)-7-methoxy-3-methyl-1,3- | |
| | dihydro-imidazo[4,5-c]quinolin-2-one | |
| | Lactose Monohydrate (Pharmatose 200) | 43.5 |
| | Microcrystalline cellulose (Vivapur 101) | 43.5 |
| | Croscarmellose Sodium | 1.00 |
| 2 | Solid preparation consisting of | |
| | 8-(1,3-Dimethyl-1H-pyrazol-4-yl)-1-(3-fluoro-5- | 10.0 |
| | methoxy-pyridin-4-yl)-7-methoxy-3-methyl-1,3- | |
| | dihydro-imidazo[4,5-c]quinolin-2-one | |
| | Lactose Monohydrate (Pharmatose 200) | 43.25 |
| | Microcrystalline cellulose (Vivapur 101) | 43.25 |
| | Crospovidone (Kollidon CL SF) | 1.0 |
| | Mg.-stearate | 0.5 |
| 3 | Solid preparation consisting of | |
| | 8-(1,3-Dimethyl-1H-pyrazol-4-yl)-1-(3-fluoro-5- | 10.0 |
| | methoxy-pyridin-4-yl)-7-methoxy-3-methyl-1,3- | |
| | dihydro-imidazo[4,5-c]quinolin-2-one | |
| | Mannitol (Parteck M100) | 43.5 |
| | Microcrystalline cellulose (Vivapur 101) | 43.5 |
| | Croscarmellose sodium | 1.0 |
| | Mg.-stearate | 1.0 |
| 4 | Solid preparation consisting of | |
| | 8-(1,3-Dimethyl-1H-pyrazol-4-yl)-1-(3-fluoro-5- | 10.0 |
| | methoxy-pyridin-4-yl)-7-methoxy-3-methyl-1,3- | |
| | dihydro-imidazo[4,5-c]quinolin-2-one | |
| | Mannitol (Parteck M100) | 42.5 |
| | Microcrystalline cellulose (Vivapur 101) | 42.5 |
| | Croscarmellose sodium | 1.0 |
| | Mg.-stearate | 2.0 |
| | Silicon dioxide | 1.0 |
| 5 | Solid preparation consisting of | |
| | 8-(1,3-Dimethyl-1H-pyrazol-4-yl)-1-(3-fluoro-5- | 10.0 |
| | methoxy-pyridin-4-yl)-7-methoxy-3-methyl-1,3- | |
| | dihydro-imidazo[4,5-c]quinolin-2-one | |
| | Mannitol (Parteck M100) | 32.5 |
| | Microcrystalline cellulose (Vivapur 101) | 42.5 |
| | Croscarmellose sodium | 1.0 |
| | Mg.-stearate | 1.0 |
| | Silicon dioxide | 1.0 |
| 6 | Solid preparation consisting of | |
| | 8-(1,3-Dimethyl-1H-pyrazol-4-yl)-1-(3-fluoro-5- | 10.0 |
| | methoxy-pyridin-4-yl)-7-methoxy-3-methyl-1,3- | |
| | dihydro-imidazo[4,5-c]quinolin-2-one | |
| | (Compound 1 Anhydrous Form A2 OPT) | |
| | Mannitol (Parteck M100) | 23.30 |
| | Microcrystalline cellulose (Vivapur 101) | 51.70 |
| | Croscarmellose sodium | 1.0 |
| | Mg.-stearate | 0.5 |
| | Silicon dioxide | 1.0 |
| 7 | Solid preparation consisting of | |
| | 8-(1,3-Dimethyl-1H-pyrazol-4-yl)-1-(3-fluoro-5- | 30.0 |
| | methoxy-pyridin-4-yl)-7-methoxy-3-methyl-1,3- | |
| | dihydro-imidazo[4,5-c]quinolin-2-one | |
| | (Compound 1 Anhydrous Form A2 OPT) | |
| | Mannitol (Parteck M100) | 13.30 |
| | Microcrystalline cellulose (Vivapur 101) | 41.70 |
| | Croscarmellose sodium | 1.0 |
| | Mg.-stearate | 0.5 |
| | Silicon dioxide | 1.0 |

Disintegration and friability test are described in the European Pharmacopoeia, Version 9.8, sections 2.9.1 (Disintegration) and section 2.9.7 (Friability of uncoated tablets).

### Example 8

The solid preparation from Examples 1 is blended for 5 min at 10 rpm. Silicon dioxide is added, and the mixture is blended for 5 min at 10 rpm. The magnesium stearate is added afterwards and the whole mixture is blended again for 2 min at 10 rpm. The whole mixture is tableted with a rotary tablet press, utilizing two pair of punches of 6 mm diameter, at compression force of 7.0 kN at a tableting speed of 20 rpm. Values for disintegration time is for a resistance to crushing of 80 N.

### Example 9

The solid preparation from Examples 2 is blended for 5 min at 10 rpm. Silicon dioxide is added, and the mixture is blended for 5 min at 10 rpm. The magnesium stearate is added afterwards and the whole mixture is blended again for 2 min at 10 rpm. The whole mixture is tableted with a rotary tablet press, utilizing two pair of punches of 6 mm diameter, at compression force of 7.0 kN at a tableting speed of 20 rpm. Values for disintegration time is for a resistance to crushing of 82 N.

### Example 10

The solid preparation from Examples 3 is blended for 5 min at 10 rpm. Silicon dioxide is added, and the mixture is blended for 5 min at 10 rpm. The mixture is tableted with a rotary tablet press, utilizing two pair of punches of 6 mm diameter, at compression force of 6.5 kN at a tableting speed of 20 rpm. Values for disintegration time is for a resistance to crushing of 85 N.

### Example 11

The solid preparation from Examples 3 is blended for 5 min at 10 rpm. Silicon dioxide is added, and the mixture is blended for 5 min at 10 rpm. The mixture is tableted with a rotary tablet press, utilizing two pair of punches of 6 mm diameter, at compression force of 6.8 kN at a tableting speed of 20 rpm. Values for disintegration time is for a resistance to crushing of 88 N.

### Example 12

The solid preparation from Examples 4 is blended for 5 min at 10 rpm. Silicon dioxide is added, and the mixture is blended for 5 min at 10 rpm. The mixture is tableted with a rotary tablet press, utilizing two pair of punches of 6 mm diameter, at compression force of 6.2 kN at a tableting speed of 20 rpm. Values for disintegration time is for a resistance to crushing of 74 N.

### Example 13

Microcrystalline cellulose and silicon dioxide are added to the solid preparation from Examples 5 and blended for 10 min at 10 rpm. Magnesium stearate is added afterwards, and the mixture is blended for 3 min at 10 rpm. The mixture is tableted with a rotary tablet press, utilizing two pair of punches of 6 mm diameter, at compression force of 4.9 kN at a tableting speed of 20 rpm. Values for disintegration time is for a resistance to crushing of 76 N.

### Example 14

Microcrystalline cellulose (Vivapur 102) and silicon dioxide are added to the solid preparation from Examples 6 and blended for 10 min at 10 rpm. Magnesium stearate is added afterwards, and the mixture is blended for 3 min at 10 rpm. The mixture is tableted with a rotary tablet press, utilizing two pair of punches of 12 mm diameter, at compression force of 23.3 kN at a tableting speed of 20 rpm. Values for disintegration time is for a resistance to crushing of 169 N.

### Example 15

Microcrystalline cellulose (Vivapur 101) and silicon dioxide are added to the solid preparation from Examples 6 and blended for 10 min at 10 rpm.

Magnesium stearate is added afterwards, and the mixture is blended for 3 min at 10 rpm. The mixture is tableted with a rotary tablet press, utilizing two pair of punches of 12 mm diameter, at compression force of 4.4 kN at a tableting speed of 20 rpm. Values for disintegration time is for a resistance to crushing of 123 N.

### Example 16

Mannitol (Parteck M200) and silicon dioxide are added to the solid preparation from Examples 7 and blended for 10 min at 10 rpm. Magnesium stearate is added afterwards, and the mixture is blended for 3 min at 10 rpm. The mixture is tableted with a rotary tablet press, utilizing two pair of punches of 12 mm diameter.

| Example/ | | | Disinte- |
|---|---|---|---|
| Formu- | | % | gration |
| lation # | Composition | (w/w) | time [s] |
| 8 | Solid preparation as listed in example #1 | 98.00 | 66 |
| | Silicon Dioxide (Aerosil 200) | 1.00 | |
| | Mg.-stearate | 1.00 | |
| | Solid preparation compressed to tablets | | |
| | and subsequently coated | | |
| 9 | Solid preparation as listed in example #2 | 98.5 | 56 |
| | Silicon Dioxide (Aerosil 200) | 1.00 | |
| | Mg.-stearate | 0.5 | |
| | Solid preparation compressed to tablets, | | |
| | potentially coated | | |
| 10 | Solid preparation as listed in example #3 | 99.0 | 103 |
| | Silicon Dioxide | 1.0 | |
| | Solid preparation compressed to tablets, | | |
| | potentially coated | | |
| 11 | Solid preparation as listed in example #3 | 98.0 | 151 |
| | Silicon Dioxide (Aerosil 200) | 2.0 | |
| | Solid preparation compressed to tablets, | | |
| | potentially coated | | |
| 12 | Solid preparation as listed in example #4 | | 178 |
| | Silicon Dioxide (Aerosil 200) | 1.0 | |
| | Solid preparation compressed to tablets, | | |
| | potentially coated | | |
| 13 | Solid preparation as listed in example #5 | 88.0 | 171 |
| | Silicon Dioxide (Aerosil 200) | 1.0 | |
| | Mg.-stearate | 1.0 | |
| | Microcrystalline Cellulose (Vivapur 101) | 10.0 | |
| 14 | Solid preparation as listed in example #6 | 87.5 | |
| | Silicon Dioxide (Aerosil 200) | 1.0 | 256 |
| | Mg.-stearate | 1.5 | |
| | Microcrystalline Cellulose (Vivapur 102) | 10.0 | |
| | Solid preparation compressed to tablets, | | |
| | potentially coated | | |
| 15 | Solid preparation as listed in example #6 | 87.5 | |
| | Silicon Dioxide (Aerosil 200) | 1.0 | 78 |
| | Mg.-stearate | 1.5 | |
| | Microcrystalline Cellulose (Vivapur 101) | 10.0 | |
| | Solid preparation compressed to tablets, | | |
| | potentially coated | | |
| 16 | Solid preparation as listed in example #7 | 88.0 | n.a. |
| | Silicon Dioxide (Aerosil 200) | 1.0 | |
| | Mg.-stearate | 1.0 | |
| | Mannitol (Parteck M200) | 10.0 | |
| | Solid preparation compressed to tablets, | | |
| | potentially coated | | |

### Exemplary capsule formulations

Disintegration test is described in the European Pharmacopoeia, Version 9.8, sections 2.9.1 (Disintegration).

### Example 17: Exemplary Capsule Formulations

8-(1 ,3-Dimethyl-1 H-pyrazol-4-yl)-1 -(3-fluoro-5-methoxy-pyridin-4-yl)-7-methoxy-3-methyl-1 ,3-dihydro-imidazo[4,5-c]quinolin-2-one is sieved trough a 0.2 mm sieve. Hardgelatin capsules (size 0, ivory) are filled with the ingredient. The disintegration of the capsule formulations is below 9 minutes.

| Example/ | | |
|---|---|---|
| Formulation # | Composition | % (w/w) |
| 17 | Solid preparation as described in | 100.00 |
| | the examples 16 | |

1. Solid preparation comprising (8-(1,3 -Dimethyl-1H-pyrazol-4-yl)- 1-(Sa)-(3 -fluoro-5-methoxy-pyridin-4-yl)-7-methoxy-3-methyl-1,3-dihydro-imidazo[4,5-c] quinolin-2-one) or a pharmaceutical acceptable salt thereof and a filler, wherein (8-(1,3-Dimethyl-1H-pyrazol-4-yl)-1-(S a)-(3 -fluoro-5-methoxy-pyridin-4-yl)-7-methoxy-3 -methyl- 1,3 -dihydro-imidazo[4,5-c]quinolin-2-one) or its pharmaceutical acceptable salt is present from 3 to 90% (w/w) based upon the total weight of the solid preparation.

2. A solid preparation according to claim 1, wherein (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) is present as its anhydrous Form A2.

3. A solid preparation according to claim 1, wherein the particle size distribution of (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) is characterized by a d10 value of at least 10 μm, a d50 value of at least 20 μm and a d90 value of at most 500 μm.

4. A solid preparation according to claim 3, whereby the ratio between the d90 value and the d10 value is in the range from 7 to 15, preferably from 8 to 14, more preferably from 9 to 13 and is most preferably about 11.

5. A solid preparation according to claim 1 4, wherein (8-(1,3 -Dimethyl-1H-pyrazol-4-yl)-1-(Sa)-(3-fluoro-5-methoxy-pyridin-4-yl)-7-methoxy-3-methyl-1,3-dihydro-imidazo[4,5-c] quinolin-2-one) present in the preparation has a monomodal particle size distribution.

6. A solid preparation according to claim 1, wherein the filler is a sugar, a sugar alcohol or dicalcium phosphate.

7. A solid preparation according to claim 6, wherein the filler is a sugar or a sugar alcohol, whereby the sugar is lactose and the sugar alcohol is sorbitol and/or mannitol, preferably mannitol.

8. A solid preparation according to claim 1, wherein the solid preparation further comprises a binder.

9. A solid preparation according to claim 8, wherein the binder is polyvinylpyrrolidone, polyvinyl acetate, a vinylpyrrolidone-vinyl acetate copolymer, polyethylene glycol, a starch paste such as maize starch paste, or a cellulose derivative such as hydroxypropyl methylcellulose, hydroxypropyl cellulose or microcrystalline cellulose, preferably microcrystalline cellulose.

10. A solid preparation according to claim 1. wherein the solid formulation further comprises a lubricant.

11. A solid preparation according to claim 10, wherein the lubricant is sodium stearyl fumarate, esters of glycerol with fatty acids, stearic acid, or pharmaceutically acceptable salts of stearic acid and divalent cations, preferably magnesium stearate.

12. A solid preparation according to claim 1. wherein the solid formulation further comprises a disintegrant.

13. A solid preparation according to claim 12, wherein the disintegrant is crospovidone, carboxy starch glycolate, cross linked carboxymethylcellulose or a salt or a derivative thereof, preferably croscarmellose sodium.

14. A solid preparation according to claim 1. wherein the solid preparation has a mean particle size that is characterized by a d50 value in the range from 20 μm to 400 μm preferably from 30 μm to 300 μm and more preferably from 40 μm to 200 μm.

15. A pharmaceutical preparation comprising the solid preparation according to claim 1.

16. A pharmaceutical preparation according to claim 15, which is a pharmaceutical preparation for oral administration.

17. A pharmaceutical preparation according to claim 15, which is an immediate release preparation.

18. A pharmaceutical preparation according to claim 15, which is a capsule comprising the solid preparation and optionally one or more pharmaceutically acceptable excipients.

19. A pharmaceutical preparation according to claim 18, which is a capsule, which contains 40 to 100% (w/w) of the solid preparation; and 0 to 60% (w/w) of at least one pharmaceutically acceptable excipient, preferably selected from a filler, a glidant, a disintegrant and a lubricant, based upon the total weight of all material contained in the capsule.

20. A pharmaceutical preparation according to claim 15, which is a tablet and which in addition to the pharmaceutically acceptable excipients present in the solid preparation optionally comprises one or more pharmaceutically acceptable excipient selected from a filler, a disintegrant, a glidant and a lubricant.

21. A pharmaceutical preparation according to claim 20, which is a tablet comprising optionally further excipients, which tablet, based upon its total weight, comprises:
i) 3 to 90% (w/w) of (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) or a pharmaceutical acceptable salt thereof;
ii) 3 to 70% (w/w) of a filler;
iii) 0 to 80% (w/w) of a binder;
iv) 0 to 20% (w/w) of disintegrant;
v) 0 to 5% (w/w) of a lubricant;
vi) 0 to 7,5% (w/w) of glidant; and
vii) a total of 0 to 20% (w/w) of one or more additional pharmaceutically acceptable excipients.

22. A pharmaceutical preparation according to claim 20, which is a tablet comprising optionally further excipients, which tablet based upon its total weight comprises:
i) 5 to 50% (w/w) of (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one);
ii) 5 to 50% (w/w) of a filler;
iii) 0 to 75% (w/w) of a binder;
iv) 0.25 to 10% (w/w) of disintegrant;
v) 0 to 4% (w/w) of a lubricant;
vi) 0 to 5% (w/w) of a glidant; and
vii) a total of 0 to 10% (w/w) of one or more additional pharmaceutically acceptable excipients.

23. A pharmaceutical preparation according to claim 20, which is a tablet comprising:
i) 7 to 30% (w/w) of (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) or a pharmaceutical acceptable salt thereof;
ii) 10 to 30% (w/w) of a filler;
iii) 10 to 60% (w/w) of a binder;
iv) 0.5 to 5% (w/w) of disintegrant;
v) 0.25 to 3% (w/w) of a lubricant;
vi) 0 to 3% (w/w) of a glidant; and
vii) a total of 0 to 10% (w/w) of one or more additional pharmaceutically acceptable excipients.

24. A pharmaceutical preparation according to claim 20, wherein the filler is mannitol , the binder is microcrystalline cellulose, the disintegrant is selected from crospovidone, carboxy starch glycolate, cross linked carboxymethylcellulose and salts and derivatives thereof, especially croscarmellose sodium, the lubricant is selected from magnesium stearate, calcium stearate and sodium stearyl fumarate, preferably magnesium stearate and/or the glidant is selected from colloidal silicon dioxide and derivatives thereof.

25. A method for preparing the solid preparation according to claim 1, the method comprising dry granulating.

26. The method for preparing the solid preparation according to claim 25, the method comprising:
(a) mixing (8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) or a pharmaceutical acceptable salt thereof and a filler and optionally one or more further pharmaceutically acceptable excipient;
(b) dry granulating the mixture prepared by step (a) to form the solid preparation; and
(c) optionally milling.

27. A method for preparing the solid preparation according to claim 25, wherein dry granulating is roller compacting.

28. A method for preparing a pharmaceutical preparation, which is a tablet, comprising a solid preparation according to claim 1, comprising
(a) conducting dry granulating to form the solid preparation;
(b) mixing the solid preparation and one or more pharmaceutically acceptable excipients;
(c) tableting the mixture prepared by step (b); and
(d) optionally film coating of the tablets prepared by step (c).

29. A method for the treatment of cancer, comprising administering to a subject in need thereof an effective amount of the pharmaceutical preparation according to claim 15.
